(19) United States
(12) Patent Application Publication
(10) Pub. No.: US 2023/0316514 A1
(43) Pub. Date: Oct. 5, 2023

(54) SYSTEM AND METHOD FOR ANGIOGRAPHIC DOSE REDUCTION USING MACHINE LEARNING

(71) Applicants: William E. Butler, Cambridge, MA (US); AngioWave Imaging, LLC, Concord, MA (US)

(72) Inventors: William E. Butler, Cambridge, MA (US); Hector Andrade-Loarca, Munich (DE)

(21) Appl. No.: 18/192,439

(22) Filed: Mar. 29, 2023

Related U.S. Application Data
(60) Provisional application No. 63/324,867, filed on Mar. 29, 2022.

(51) Int. Cl. G06T 7/00; A61B 6/00; G06T 7/11; G06T 7/174

(52) U.S. Cl. CPC G06T 7/0012; A61B 6/481; A61B 6/504; A61B 6/5235; A61B 6/508; G06T 7/11; G06T 7/174; G06T 2207/30104; G06T 2207/20081; G06T 2207/10121; G06T 2207/10016

(57) ABSTRACT

Methods, systems, and computer readable media are provided for reduced-dose angiography using machine learning (e.g., deep learning). Briefly, techniques described herein may use a neural network trained to conserve/preserve angiographic image quality while reducing the angiographic dose of potentially harmful chemical contrast and/or x-ray radiation. As a result, angiographic anatomy may be extracted from an image at reduced angiographic doses using a deep learning neural network. The reduction in chemical contrast and x-ray dose may be achieved based on operations performed before, during, and/or after angiographic imaging.

### CROSS-REFERENCE TO RELATED APPLICATION

This application claims priority to U.S. Provisional Patent Application No. 63/324,867, filed on Mar. 29, 2022, the disclosure of which is incorporated by reference herein in its entirety.

### FIELD

The present disclosure relates generally to angiography, and more specifically to a system and method for performing angiography with dose reduction using machine learning.

### BACKGROUND

The heart sends blood as a sequence of arterial stroke volumes throughout the body, where the blood crosses the capillaries to the veins and returns to the heart. The presence and motions of blood in the blood vessels (generally, branching tubular structures) can be dynamically imaged with a technique called angiography.

In fluoroscopic x-ray angiography, a chemical contrast agent is injected into the vascular system (e.g., blood stream) as a bolus in coordination with obtaining a sequence of x-ray images. The chemical contrast agent may include any one or more of a plurality of chemical substances in liquid form. The chemical contrast agent is denser than blood or tissue; therefore, the chemical contrast agent attenuates the passage of x-rays more than blood or tissue. The denser the agent, the sharper the image contrast imparted on the containing vessel during fluoroscopic angiographic imaging. Some particularly dense formulations of chemical contrast agent contain iodine in its ionic form; this class of agents is termed “iodinated contrast.” One example of iodinated contrast is iohexol. Other chemical contrast agents do not have iodine and are not iodinated.

The chemical contrast agent travels through the vasculature, blocking the passage of the x-rays at a given frame rate and creating an impression of the contrast-containing vascular structures. The resulting spatiotemporal x-ray attenuation pattern creates a sequence of x-ray images fluoroscopically obtained on an x-ray sensor. The sequence may be referred to as an angiogram, e.g., a sequence of images that trace the passage of the bolus of contrast. Angiograms are typically two-dimensional in space and one-dimensional in time.

The injected chemical contrast agent has toxic side effects to kidneys and other internal organs. However, lowering the dose of contrast agent to reduce the risk of these toxic side effects may produce unsatisfactory images with poor signal to noise ratios which, in turn, may lead to incomplete angiographic studies with inadequately imaged vascular anatomy. Use of a lower dose of contrast agent may also compel the advancement of the injecting catheter further into the arterial tree so that the injected contrast remains concentrated within the anatomic region of interest. The need to advance the injecting catheter further elevates the risk of complications caused by the catheter injuring ever smaller vessels distal in the vascular tree.

### SUMMARY

Aspects of the invention are directed to methods, systems, and computer readable media for angiographic dose reduction using machine learning (e.g., deep learning). Briefly, techniques described herein use machine learning to conserve/preserve angiographic image quality while allowing the angiographic dose of potentially harmful chemical contrast and/or x-ray radiation to be reduced. As a result, angiographic anatomy may be extracted from an image at reduced angiographic doses. The reduction in chemical contrast and/or x-ray dose may be achieved based on operations performed before, during, and/or after angiographic imaging.

Various mechanisms are provided to train a machine learning model (e.g., a deep learning neural network) to produce higher-quality angiographic images with a reduced dosage of chemical contrast agent and/or x-ray radiation. These mechanisms may involve gathering training data acquired with a reduced dose of chemical contrast agent and/or x-ray radiation (“reduced-dose training data”) crossed with standard full-dose data that has been segmented (“full-dose segmented data”). That is, a training data set may be generated using full-dose segmented data acquired at full (standard) doses of chemical contrast and x-ray radiation as known outputs, and non-segmented reduced-dose training data, to train the neural network against the full-dose segmented data. The training data set may be generated using: full (standard) doses and physically reduced doses in animal (e.g., non-human) angiograms; full (standard) doses and physically reduced doses in realistic artificial models of organs; and/or by computationally simulated reduction of doses in full dose human angiographic data. Thus, in a neural network training step, a plurality of angiographic images acquired at full (standard) doses across a temporal interval may be employed to segment blood vessels, and a neural network (e.g., a convolutional network) may obtain the standard dose segmentation results with reduced dose image data. The neural network may thus be trained against training data with high-quality angiographic structure ground-truth data and actual or simulated degraded angiographic images captured with reduced chemical contrast and/or x-ray radiation doses.

In a neural network post-training deployment mode, the neural network may obtain angiographic data acquired at reduced chemical contrast and/or x-ray radiation doses to produce an image segmentation based on its training against full dose segmentations. In one example, the lower-quality angiographic image data from physically reduced chemical contrast and/or x-ray radiation doses may be supplied to the deep neural network to estimate vascular structures according to the training of reduced quality angiographic images against higher quality ground truth training data. Thus, the deep learning neural network may produce vascular segmentation of images acquired at physically reduced chemical and/or x-ray radiation doses.

The neural network may be applied to a span or sequence of angiographic images to identify vascular structure without increased angiographic doses. For example, the deep learning neural network may estimate the vascular structures in a target angiographic image using the target angiographic image and one or more temporally adjacent or contiguous angiographic images. In this way, the neural network may be tuned to detect spatiotemporal properties of contrast-containing blood vessels.

Separate neural network configurations may be designed to optimize offline calculations and/or real-time calculations. For example, the neural network may be modified for real-time uses where the plurality of angiographic images for data input include a plurality of recent temporally preceding angiographic images for producing a segmentation for the most recent angiographic image.

In accordance with a first aspect of the invention, a method comprises providing, as an input to a machine learning model, via a processor, a target angiographic image obtained using a first dose of a chemical contrast agent and/or x-ray radiation, the machine learning model having been trained using (a) a second angiographic image obtained using a second dose of a chemical contrast agent and/or x-ray radiation as an input, and (b) a third angiographic image obtained using a third dose of a chemical contrast agent and/or x-ray radiation as a known output, and wherein the third dose is greater than the first and second doses. The method further comprises obtaining, from the machine learning model, via the processor, an output comprising an angiographic image that is a processed version of the target angiographic image. Processing a first angiographic image obtained with a first dose of a chemical contrast agent and/or x-ray radiation using a machine learning model trained with a second angiographic image obtained using a second dose of a chemical contrast agent and/or x-ray radiation as training data and a third angiographic image obtained using a third dose of a chemical contrast agent and/or x-ray radiation as a known output, wherein the third dose is greater than the first dose, may improve the safety of an angiographic study by allowing diagnostically useful angiographic images to be obtained from angiographic images acquired with reduced doses of chemical contrast agent and/or x-ray radiation.

In an embodiment, the target image is one of a plurality of angiographic images in a first sub-sequence of angiographic images obtained using the first dose of a chemical contrast agent and/or x-ray radiation and provided to the machine learning model as an input, and the second angiographic image is one of a plurality of angiographic images in a second sub-sequence of angiographic images obtained using the second dose of a chemical contrast agent and/or x-ray radiation and used to train the machine-learning model. By providing a sub-sequence of angiographic images obtained with a first dose of a chemical contrast agent and/or x-ray radiation and containing the target image to a machine learning model trained with a sub-sequence of angiographic images obtained using a second dose of a chemical contrast agent and/or x-ray radiation, this embodiment may improve the accuracy of the output by using spatiotemporal information from other images in the sub-sequence of images containing the target image.

In an embodiment, the first sub-sequence of angiographic images is provided to the machine learning model as a single vector. By providing the first sub-sequence of angiographic images to the machine learning model as a single vector, this embodiment may reduce processing time.

In an embodiment, the first and second sub-sequences of angiographic images each consist of an odd number of angiographic images. Using an odd number of angiographic images in each sub-sequence may improve accuracy of the output by allowing the target image to be taken from a middle of the first sub-sequence of angiographic images and providing spatiotemporal information from before and after the target image.

In an embodiment, the first subsequence of angiographic images consists of a same number of angiographic images as the second sub-sequence of angiographic images. By ensuring that the input in the deployment mode is consistent with the input in the training mode, this embodiment may reduce the time needed to train the machine learning model.

In an embodiment, the first and second sub-sequences each consist of five angiographic images, which is advantageous in that the target image may be located in the middle of the sub-sequence such that spatiotemporal information before and after the target image is available. It has been found that this embodiment can provide diagnostically useful output with less processing than sub-sequences greater than five images.

In an embodiment, the first sub-sequence of angiographic images is one of a plurality of input sub-sequences, wherein each of the plurality of input sub-sequences was extracted from a different part of a sequence of angiographic images obtained using the first dose of a chemical contrast agent and/or x-ray radiation. The plurality of input sub-sequences may be provided to the machine learning model as inputs to obtain from the machine learning model, via the at least one processor, outputs comprising angiographic images that are processed versions of respective target angiographic images in each of the plurality of input sub-sequences. This embodiment allows multiple target images in a sequence of images to be processed using the machine learning model, which may be advantageous for purposes of diagnosis.

In an embodiment, the second sub-sequence of angiographic images is one of a plurality of sub-sequences used to train the machine-learning model, and wherein each of the plurality of sub-sequences was extracted from a different part of a sequence of angiographic images obtained using the second dose of a chemical contrast agent and/or x-ray radiation. This embodiment allows the machine learning model to be trained to process multiple target images in a sequence of images, which may be advantageous for purposes of diagnosis.

In an embodiment, each of the angiographic images in the first sub-sequence of angiographic images is temporally contiguous with at least one other angiographic image in the first sub-sequence, and each of the angiographic images in the second sub-sequence of angiographic images is temporally contiguous with at least one other angiographic image in the second sub-sequence. Using temporally contiguous images may improve the accuracy of the output by providing spatiotemporal information from images the target image.

In an embodiment, the first and second doses of chemical contrast agent and/or x-ray radiation are the same. This embodiment may improve accuracy of the output.

In an embodiment, the first and second doses are less than a dose required to obtain diagnostically useful images from an x-ray imaging device, and the third dose is no less than the dose required to obtain diagnostically useful images from the x-ray imaging device. This embodiment may improve the safety of an angiographic study by allowing angiographic images to be obtained with reduced doses of chemical contrast agent and/or x-ray radiation.

In an embodiment, the angiographic images in the second sub-sequence are angiographic images of non-human vascular structures. This embodiment may reduce the cost of training the machine learning model.

In an embodiment, the angiographic images in the second sub-sequence are angiographic images of artificial vascular structures. This embodiment may reduce the cost of training the machine learning model.

In an embodiment, the second dose of chemical contrast agent and/or x-ray radiation is greater than the first dose of chemical contrast agent and/or x-ray radiation, and the angiographic images in the second sub-sequence are angiographic images that have been modified to simulate having been obtained using less than the second dose of chemical contrast agent and/or x-ray radiation. This embodiment allows more control over the quality of the training data and may reduce the cost of training the machine learning model.

In an embodiment, the second dose of chemical contrast agent and/or x-ray radiation is greater than the first dose of chemical contrast agent and/or x-ray radiation, and the second angiographic image is modified to simulate having been obtained using less than the second dose of chemical contrast agent and/or x-ray radiation. This embodiment allows more control over the quality of the training data and may reduce the cost of training the machine learning model.

In an embodiment, the second angiographic image is modified by adding randomly generated noise to at least some pixels of the second angiographic image. This embodiment allows more control over the quality of the training data and may reduce the cost of training the machine learning model.

In an embodiment, the randomly generated noise is added only to pixels of the second angiographic image that are determined to correspond to vessels. This embodiment reduces cost of training the machine learning model and helps avoid registration errors due to movement of organs in the angiographic field before and after the chemical contrast agent is administered.

In an embodiment, the third angiographic image is a segmented angiographic image, and the output obtained from the machine learning model is an angiographic image that is a segmented version of the first angiographic image. This embodiment may decrease processing time by eliminating the need for a separate manual, semi-automated, or automated segmentation step.

In an embodiment, the processed version of the first angiographic image may be displayed on a display device. This embodiment allows a medical professional to view and analyze the output in order to provide a diagnosis or other medical opinion.

Other aspects of the invention include a system and a computer program product utilizing substantially the same techniques described above.

Other objects and advantages of these techniques will be apparent from the specification and drawings.

### BRIEF DESCRIPTION OF THE DRAWINGS

FIGS. 1A and 1B are side and partially schematic views, respectively, showing an example of a rotational x-ray system that may be used with embodiments of the disclosure for acquiring angiographic data.

FIG. 2 is a schematic diagram of a computer system or information processing device that may be used with embodiments of the disclosure.

FIGS. 3A-3C each illustrate a flowchart of a method for training a neural network based on respective training data, according to an example embodiment.

FIG. 4 illustrates a system configured to generate simulated images of vascular structures acquired at lower doses of chemical contrast and/or x-ray radiation, according to an example embodiment.

FIG. 5 illustrates an example method for segmenting vasculature objects in a single image from an angiogram acquired at low chemical contrast and/or x-ray doses, according to an example embodiment.

FIG. 6 illustrates generating a higher-quality angiographic image from a target angiographic image that is located in various positions within a sub-sequence of angiographic images, using machine learning according to an example embodiment.

### DETAILED DESCRIPTION OF EXAMPLE EMBODIMENTS

Fluoroscopic angiographic imaging with chemical contrast is a commonly employed medical imaging procedure. Briefly, a physician secures access percutaneously into a blood vessel (commonly the femoral artery), navigates a catheter to the root artery of the organ in question, and injects the chemical contrast in temporal coordination with the fluoroscopic acquisition of a sequence of x-ray images.

Fluoroscopic angiography may be used to diagnose blocked coronary arteries of the heart or in the brain. The same angiographic study may offer an avenue for therapy if that study establishes the diagnosis of a blocked artery. For example, a stent may be placed across a calcified plaque in a coronary artery of the heart, or a thrombolytic medication may be applied directly to a blood clot in a cerebral artery.

An angiographic imaging study may include target objects in the foreground, such as blood vessels. The greater the sharpness, detail, and clarity with which foreground objects in the imaging study are displayed relative to the background, the greater the signal to noise ratio of the imaging study and, therefore, the greater the diagnostic value. But in some cases, fluoroscopic angiography can provide insufficient clarity to the configuration of blood vessels in an angiographic image, even when chemical contrast is injected to improve definition of the blood vessels in the anatomic area being fluoroscopically imaged.

In standard practice, the image quality of the angiographic study can be improved by increasing the dose of the injected chemical contrast and/or by increasing the dose of the fluoroscopic x-ray radiation. However, use of chemical contrast and x-ray radiation in an angiographic imaging study can carry risk. For example, there is risk associated with the procedure of placing a catheter into a blood vessel and navigating it to the organ of interest for chemical contrast injection. In addition, the chemical contrast agent and the x-ray radiation can be harmful to (e.g., cause toxic side-effects in) a human or animal study subject.

All chemical contrast agents can have a measure of toxicity when injected into the vascular circulation system. Upon injection, the chemical contrast agent may begin to be cleared by biochemical and physiological processes, led by clearing organs such as the kidneys and the liver. The chemical contract agent may be toxic to the clearing organs in a dose-dependent manner.

The chemical contrast agent can also produce a significant mass load into the vascular system, producing stress on the heart and other vascular structures, potentially inducing or aggravating heart failure in a patient with compromised heart pump action. The agent can also place stress on the clearing organs.

Also, some humans may develop immune reactions to specific molecular structures on various chemical contrast agents, particularly on iodinated contrast agents. A histiocyte-mediated immune reaction to an injected contrast agent may be immediate and severe, and can be fatal if not promptly recognized and treated. For at least these reasons, increasing the chemical contrast to obtain a higher-quality angiography study also increases the risk of harm to the subject.

In addition, x-ray doses can be harmful to irradiated tissues, particularly in tissues that are particularly sensitive to radiation, such as the thyroid gland and reproductive organs. Radiation doses may induce chronic inflammation and/or injure constituent bio-molecules of tissues, potentially leading to consequences ranging from comparatively minor skin irritation along the x-ray path to cancer formation at irradiated tissues. Higher x-ray doses in particular can directly injure the bio-molecular constituents of tissue, including deoxyribonucleic acid (DNA) in the cell nuclear apparatus, producing malformations of organs and carrying a risk of neoplasia.

Accordingly, techniques are described herein to reduce the dosage of chemical contrast and/or x-ray radiation that are administered during an angiographic procedure. In particular, angiographic image quality may be conserved by displaying the imaged vasculature at greater sharpness and accuracy in the foreground using lower/reduced doses both of chemical contrast and x-ray radiation. As described in greater detail below, this may be accomplished using machine learning (e.g., deep learning) techniques.

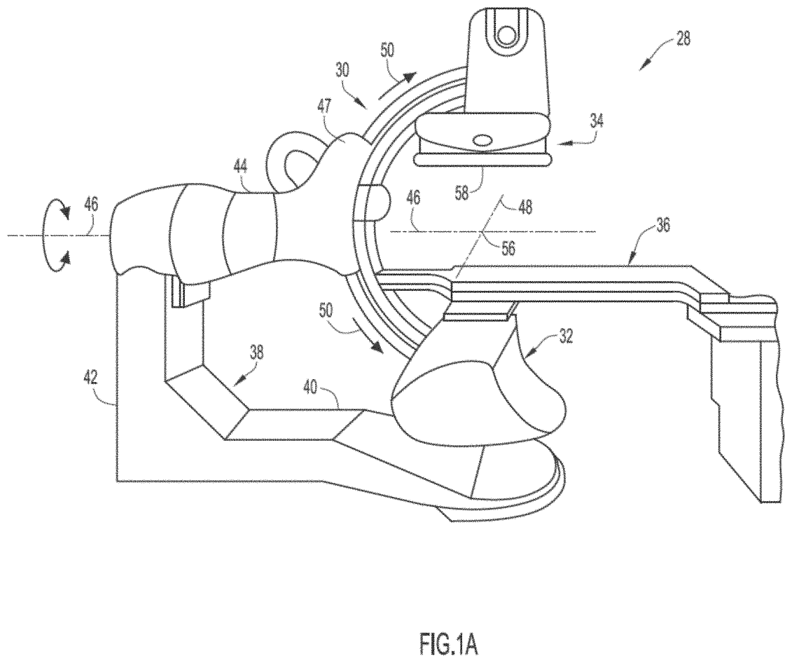

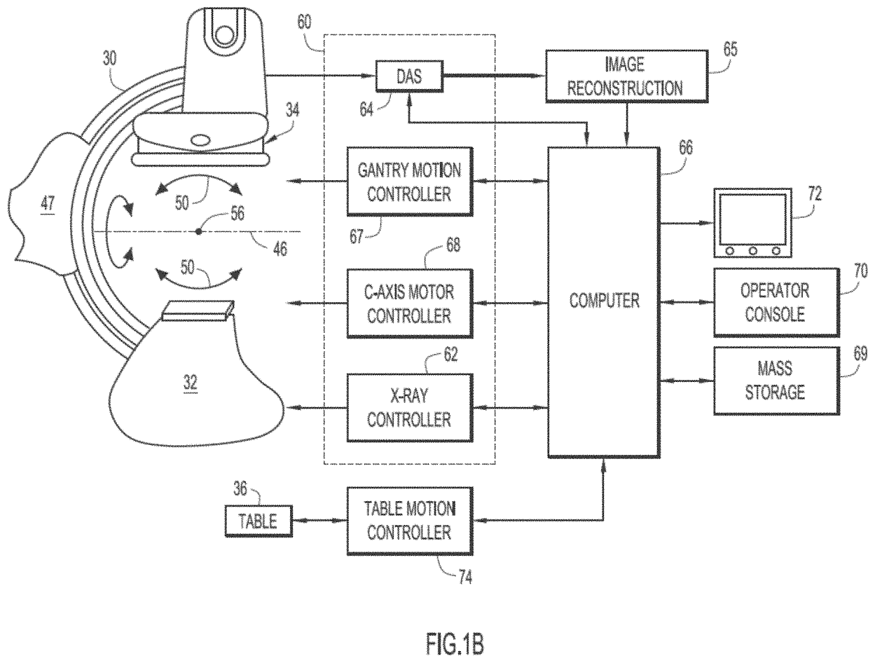

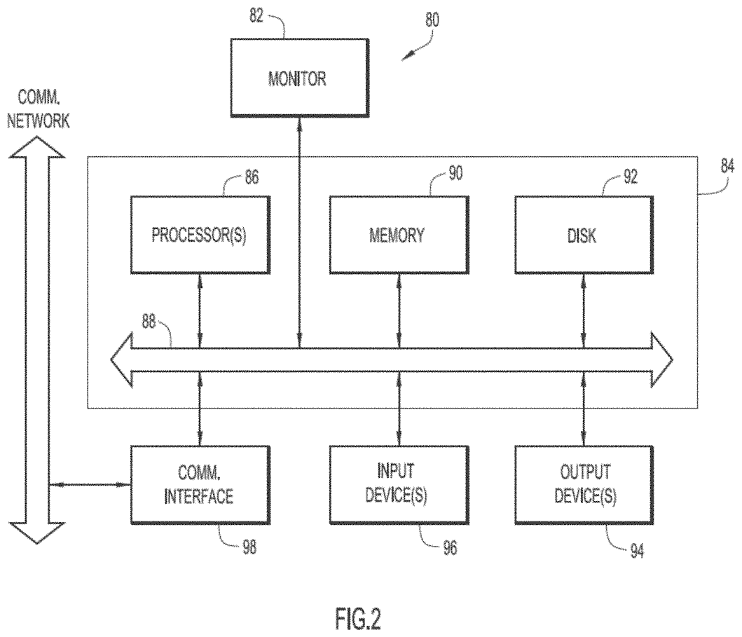

Referring to FIGS. 1A, 1B, and 2, exemplary systems or devices that may be employed for carrying out embodiments of the invention are illustrated. It is understood that such systems and devices are only exemplary of representative systems and devices and that other hardware and software configurations are suitable for use with embodiments of the invention. Thus, the embodiments are not intended to be limited to the specific systems and devices illustrated herein, and it is recognized that other suitable systems and devices can be employed without departing from the spirit and scope of the subject matter provided herein.

Referring first to FIGS. 1A and 1B, a rotational x-ray system 28 is illustrated that may be employed for obtaining an angiogram via fluoroscopic angiography. In acquiring an angiogram, a chemical contrast agent may be injected into the patient positioned between an x-ray source and detector, and x-ray projections are captured by the x-ray detector as a two-dimensional image (i.e., an angiographic image or image frame). A sequence of such image frames comprises an angiographic study. In one example, the sequence of angiographic image frames may be obtained at a rate faster than the subject's cardiac rate. For example, the subject's cardiac rate may be measured (e.g., using an EKG device or the like) and the sequence of angiographic image frames may be obtained at a rate faster than the measured cardiac rate.

As shown in FIG. 1A, an example of an angiogram imaging system is shown in the form of a rotational x-ray system 28 including a gantry having a C-arm 30 which carries an x-ray source assembly 32 on one of its ends and an x-ray detector array assembly 34 at its other end. The gantry enables the x-ray source assembly 32 and x-ray detector array assembly 34 to be oriented in different positions and angles around a patient disposed on a table 36, while providing to a physician access to the patient. The gantry includes a pedestal 38 which has a horizontal leg 40 that extends beneath the table 36 and a vertical leg 42 that extends upward at the end of the horizontal leg 40 that is spaced apart from table 36. A support arm 44 is rotatably fastened to the upper end of vertical leg 42 for rotation about a horizontal pivot axis 46.

The horizontal pivot axis 46 is aligned with the centerline of the table 36, and the support arm 44 extends radially outward from the horizontal pivot axis 46 to support a C-arm drive assembly 47 on its outer end. The C-arm 30 is slidably fastened to the C-arm drive assembly 47 and is coupled to a drive motor (not shown) which slides the C-arm 30 to revolve about a C-axis 48 as indicated by arrows 50. The horizontal pivot axis 46 and C-axis 48 intersect each other, at a system isocenter 56 located above the table 36, and are perpendicular to each other.

The x-ray source assembly 32 is mounted to one end of the C-arm 30 and the x-ray detector array assembly 34 is mounted to its other end. The x-ray source assembly 32 emits a beam of x-rays which are directed at the x-ray detector array assembly 34. Both assemblies 32 and 34 extend radially inward to the horizontal pivot axis 46 such that the center ray of this beam passes through the system isocenter 56. The center ray of the beam thus can be rotated about the system isocenter around either the horizontal pivot axis 46 or the C-axis 48, or both, during the acquisition of x-ray attenuation data from a subject placed on the table 36.

The x-ray source assembly 32 contains an x-ray source which emits a beam of x-rays when energized. The center ray passes through the system isocenter 56 and impinges on a two-dimensional flat panel digital detector 58 housed in the x-ray detector array assembly 34. The two-dimensional flat panel digital detector 58 may be, for example, a 2048×2048 element two-dimensional array of detector elements. Each element produces an electrical signal that represents the intensity of an impinging x-ray and hence the attenuation of the x-ray as it passes through the patient. During a scan, the x-ray source assembly 32 and x-ray detector array assembly 34 are rotated about the system isocenter 56 to acquire x-ray attenuation projection data from different angles. In some embodiments, the detector array is able to acquire fifty projections, or image frames, per second which is the limiting factor that determines how many image frames can be acquired for a prescribed scan path and speed.

Referring to FIG. 1B, the rotation of the assemblies 32 and 34 and the operation of the x-ray source are governed by a control mechanism 60 of the x-ray system. The control mechanism 60 includes an x-ray controller 62 that provides power and timing signals to the x-ray source assembly 32. A data acquisition system (DAS) 64 in the control mechanism 60 samples data from detector elements and passes the data to image reconstruction system or module 65. The image reconstruction system 65 receives digitized x-ray data from the DAS 64 and performs high speed image reconstruction according to the methods of the present disclosure. The reconstructed image is applied as an input to a computer 66 which stores the image in a mass storage device 69 or processes the image further. Image reconstruction system 65 may be included in a standalone computer or may be integrated with computer 66.

The control mechanism 60 also includes gantry motor controller 67 and a C-axis motor controller 68. In response to motion commands from the computer 66, the motor controllers 67 and 68 provide power to motors in the x-ray system that produce the rotations about horizontal pivot axis 46 and C-axis 48, respectively. The computer 66 also receives commands and scanning parameters from an operator via console 70 that has a keyboard and other manually operable controls. An associated display 72 allows the operator to observe the reconstructed image frames and other data from the computer 66. The operator supplied commands are used by the computer 66 under the direction of stored programs to provide control signals and information to the DAS 64, the x-ray controller 62 and the motor controllers 67 and 68. In addition, computer 66 operates a table motor controller 74 which controls the motorized table 36 to position the patient with respect to the system isocenter 56.

Referring now to FIG. 2, a block diagram of a computer system or information processing device 80 (e.g., image reconstruction system 65 and/or computer 66 in FIG. 1B) is illustrated that may be incorporated into an angiographic imaging system, such as the rotational x-ray system 28 of FIGS. 1A and 1B, and/or may be used as a standalone device, for angiographic dose reduction using machine learning according to embodiments of the present invention. Information processing device 80 may be local to or remote from rotational x-ray system 28. In one example, the functionality performed by information processing device 80 may be offered as a Software-as-a-Service (SaaS) option. SaaS refers to a software application that is stored in one or more remote servers (e.g., in the cloud) and provides one or more services (e.g., angiographic image processing) to remote users. Angiographic images may be obtained directly from an angiographic imaging system, such as the rotational x-ray system 28 of FIGS. 1A and 1B, or from other sources such as physical storage media configured to store data.

In one embodiment, computer system 80 includes a monitor or display device 82, a computer system 84 (which includes processor(s) 86, bus subsystem 88, memory subsystem 90, and disk subsystem 92), user output devices 94, user input devices 96, and communications interface 98. Monitor 82 can include hardware and/or software elements configured to generate visual representations or displays of information. Some examples of monitor 82 may include familiar display devices, such as a television monitor, a cathode ray tube (CRT), a liquid crystal display (LCD), a light-emitting diode (LED) display, or the like. In some embodiments, monitor 82 may provide an input interface, such as incorporating touch screen technologies.

Computer system 84 can include familiar computer components, such as one or more central processing units (CPUs), memory or storage devices, graphics processing units (GPUs), communication systems, interface cards, or the like. As shown in FIG. 2, computer system 84 may include at least one hardware processor 86 that communicates with a number of peripheral devices via bus subsystem 88. Processor(s) 86 may include commercially available central processing units or the like. Bus subsystem 88 can include mechanisms for letting the various components and subsystems of computer system 84 communicate with each other as intended. Although bus subsystem 88 is shown schematically as a single bus, alternative embodiments of the bus subsystem may utilize multiple bus subsystems. Peripheral devices that communicate with processor(s) 86 may include memory subsystem 90, disk subsystem 92, user output devices 94, user input devices 96, communications interface 98, or the like.

Processor(s) 86 may be implemented using one or more analog and/or digital electrical or electronic components, and may include a microprocessor, a microcontroller, an application-specific integrated circuit (ASIC), a field programmable gate array (FPGA), programmable logic and/or other analog and/or digital circuit elements configured to perform various functions described herein, such as by executing instructions stored in memory subsystem 90 and/or disk subsystem 92 or another computer program product.

Memory subsystem 90 and disk subsystem 92 are examples of physical storage media configured to store data, such as instructions executable by the one or more processors 86 to perform the operations described herein. Memory subsystem 90 may include a number of memories or memory devices including random access memory (RAM) for volatile storage of program code, instructions, and data during program execution and read only memory (ROM) in which fixed program code, instructions, and data are stored. Disk subsystem 92 may include a number of file storage systems providing persistent (non-volatile) storage for programs and data. Other types of physical storage media include floppy disks, removable hard disks, optical storage media such as compact disc—read-only memories (CD-ROMS), digital video disc (DVDs) and bar codes, semiconductor memories such as flash memories, read-only-memories (ROMS), battery-backed volatile memories, networked storage devices, or the like. Memory subsystem 90 and disk subsystem 92 may be configured to store programming and data constructs that provide functionality or features of techniques discussed herein. Software code modules and/or processor instructions that when executed by processor(s) 86 implement or otherwise provide the functionality may be stored in memory subsystem 90 and disk subsystem 92. Memory subsystem 90 may be a non-transitory computer readable storage medium.

User input devices 96 can include hardware and/or software elements configured to receive input from a user for processing by components of computer system 80. User input devices can include all possible types of devices and mechanisms for inputting information to computer system 84. These may include a keyboard, a keypad, a touch screen, a touch interface incorporated into a display, audio input devices such as microphones and voice recognition systems, and/or other types of input devices. In various embodiments, user input devices 96 may include a computer mouse, a trackball, a track pad, a joystick, a wireless remote, a drawing tablet, a voice command system, an eye tracking system, or the like. In some embodiments, user input devices 96 are configured to allow a user to select or otherwise interact with objects, icons, text, or the like that may appear on monitor 82 via a command, motions, or gestures, such as a click of a button or the like.

User output devices 94 can include hardware and/or software elements configured to output information to a user from components of computer system 80. User output devices can include all possible types of devices and mechanisms for outputting information from computer system 84. These may include a display device (e.g., monitor 82), a printer, a touch or force-feedback device, audio output devices, or the like.

Communications interface 98 can include hardware and/or software elements configured to provide unidirectional or bidirectional communication with other devices. For example, communications interface 98 may provide an interface between computer system 84 and other communication networks and devices, such as via an internet connection.

Techniques described herein may enable reduction of the angiographic doses (e.g., chemical contrast agent doses and/or x-ray radiation doses) required to obtain a diagnostically useful angiographic image. In particular, the angiographic doses may be reduced compared to standard/conventional angiographic doses that would otherwise be required to obtain a diagnostically useful angiographic image in the absence of the techniques described herein (e.g., in the absence of a machine learning model, such as a deep learning neural network, as described herein). In one example, a “diagnostically useful” or “high-quality” angiographic image provides data of a quality sufficient to provide meaningful clinical information and/or to allow treatment decisions to be made, e.g., a diagnostically useful angiographic image may be one of sufficient clarity to allow health care professionals to visually identify and segment vessels in the image. For example, if a person has chest pain due to insufficient blood flow to the coronary arteries that supply blood to the heart muscle, a diagnostically useful angiographic image of the coronary arteries may accurately display the segment of the coronary artery of the heart with a stenosis that impairs circulation to the heart muscle.

The x-ray dosage required to generate a diagnostically useful image in an angiogram also varies depending on physical characteristics of the patient/subject and the nature of the angiographic procedure. Methods of calculating x-ray dosages are well known in the art. Typically, a full or standard dose of x-ray radiation for an interventional cardiac procedure in a fluoroscopic unit ranges from 8 to 10 milliSieverts of radiation. A Sievert is the equivalent of 1 Joule of energy per kilogram of mass. The ionizing nature of x-ray radiation means that it is always desirable to minimize the exposure of the subject (and medical staff associated with an angiography procedure) to x-rays as much as possible while still producing a useful visualization of the target tissue.

The main type of contrast agent used in angiography is the family of iodinated contrast agents, which can be ionic or, advantageously, non-ionic iodinated contrast agents. Such contrast agents are well known in the art and include: iohexol (Omnipaque™, GE Healthcare); iopromide (Ultravist™, Bayer Healthcare); iodixanol (Visipaque™, GE Healthcare); ioxaglate (Hexabrix™ Mallinckrodt Imaging); iothalamate (Cysto-Conray II™, Mallinckrodt Imaging); and iopamidol (Isovue™, Bracco Imaging). See also Lusic and Grinstaff, “X-Ray Computed Tomography Contrast Agents,” Chem Rev. 13:1641-66 (2013). Examples of other agents include gadolinium-based agents. See Ose et al., “‘Gadolinium’ as an Alternative to Iodinated Contrast Media for X-Ray Angiography in Patients With Severe Allergy,” Circ J. 2005; 69:507-509 (2005).

The standard or full-dosages for such chemical contrast agents vary depending on the nature of the agent, the physical characteristics of the patient/subject, and the nature of the angiographic procedure. In general, however, the standard or full-dosages of such chemical contrast agents improve the visualization of the target tissue by increasing the difference in absolute CT (computerized tomography) attenuation value between the target tissue and surrounding tissue and fluids. For fluoroscopic angiography, the injected chemical contrast agent typically increases the CT attenuation value of a blood vessel somewhere between 2× and 10× the baseline level without the chemical contrast agent. In fluoroscopic angiography, the injection catheter is navigated near to the target organ, allowing a higher local dose but a lower total body dose. In CT, where the contrast is injected intravenously, the contrast is diluted throughout the vascular system. Thus, the local concentration of contrast in any given blood vessel may be lower. The increase in CT attenuation value from such an intravenous injection of a chemical contrast agent is typically between 1.2× and 4× the baseline CT attenuation value without the chemical contrast agent. An increase of at least 1.2× in CT attenuation from baseline for CT angiography, and at least 2× in CT attenuation from baseline for fluoroscopic angiography, have been found to provide “diagnostically useful” or “high-quality” angiographic images. The contrast agent should preferably contain a high mol % of the x-ray attenuating atom per agent (molecule, macromolecule, or particle) in order to reduce the volume used and concentrations needed for imaging. Also, the tissue retention-time of the contrast agent should preferably be sufficiently long for completion of a CT scan and scheduling the instrument time in the diagnostic setting (e.g., 2-4 h). Moreover, the contrast agent preferably should: (a) localize or target the tissue of interest and possess favorable biodistribution and pharmacokinetic profiles; (b) be readily soluble or form stable suspensions at aqueous physiological conditions (appropriate pH and osmolality) with low viscosity; (c) be non-toxic; and (d) be cleared from the body in a reasonably short amount of time, usually within several hours (<24 h).

In some embodiments, the angiographic iodinated contrast dose used to obtain diagnostically useful angiographic images may be reduced by about 25% of the standard or full-dosage amount of contrast that is typically injected. Further dose reduction may be achieved using the techniques described herein in combination with spatiotemporal reconstruction techniques as described in U.S. application Ser. No. 16/784,073, filed on Feb. 6, 2020, which is incorporated by reference herein in its entirety. The spatiotemporal reconstruction of an image may be an input to the machine learning model, or the output of the machine learning model may be processed using spatiotemporal reconstruction.

To increase the sharpness and clarity of the imaged vasculature at lower doses of chemical contrast and x-ray radiation, a deep learning neural network (e.g., having an input layer, an output layer, and three or more layers between the input and output layers) is provided with properties that promote its performance in detecting vasculature at low chemical contrast and/or x-ray doses. While descriptions provided herein focus on deep learning neural networks, it will be appreciated that these techniques may be utilized with any suitable machine learning model, of which a deep learning neural network is just one example. The machine learning model may be implemented by any suitable machine learning techniques (e.g., mathematical/statistical, classifiers, feed-forward, recurrent, convolutional or other neural networks, etc.). For example, a neural network may be used that includes an input layer, one or more intermediate layers (e.g., including any hidden layers), and an output layer. Each layer may include one or more nodes or neurons, where the input layer neurons receive input (e.g., image data, feature vectors of images, etc.), and may be associated with weight values. For example, each node in the input layer may receive or be encoded with the relative brightness of one pixel of an angiographic image as an input, and the relative brightness may be a floating point number between 0 and 1. The neurons of the intermediate and output layers are connected to one or more neurons of a preceding layer, and receive as input the output of a connected neuron of the preceding layer. Each connection is associated with a weight value, and each neuron produces an output based on a weighted combination of the inputs to that neuron. The output of a neuron may further be based on a bias value for certain types of neural networks (e.g., recurrent types of neural networks). The weight (and bias) values may be adjusted based on various training techniques. For example, the machine learning of the neural network may be performed using a training set of reduced-dosage angiographic images as input and corresponding high-quality full-dosage angiographic images as known outputs, where the neural network attempts to produce the known output by using an error from the output produced by the neural network (e.g., difference between produced and known outputs) to adjust weight (and bias) values (e.g., via backpropagation or other training techniques). In example embodiments, the known output at each node may be a pixel value representing brightness or intensity (e.g., a floating point number between 0 and 1) or a probability (p-value) that the pixel is a vessel (e.g., a floating point number between 0 and 1). A neural network trained using the latter technique (i.e., wherein the known output is a segmented angiographic image with p-values of 0-1 for each pixel) can be advantageously used to produce a segmented angiographic image from reduced-dosage angiographic images.

More specifically, in a training phase, a data conditioning system may be provided to train the deep learning neural network to perform well in the setting of low chemical contrast and/or x-ray doses. Thus, the training phase may enable a deployment phase in which the deep learning neural network is able to output high-quality angiographic images based on angiographic images obtained at lower/reduced chemical contrast and/or x-ray radiation doses.

In one aspect, a deep learning network data training system is provided that promotes the ability of a convolutional spatiotemporal network to detect pixels corresponding to blood vessels despite the use of low doses of chemical contrast and/or fluoroscopic x-ray radiation. The deep learning neural network may be trained using angiographic training data. The angiographic training data may include (1) a first set of angiographic images obtained at conventional/standard chemical contrast and x-ray radiation doses, and (2) comparable angiographic images with reduced chemical contrast and/or x-ray radiation doses. In an embodiment, the comparable angiographic images with reduced chemical contrast and/or x-ray radiation doses may be used for the training set as inputs, and the angiographic images obtained at conventional/standard chemical contrast and x-ray radiation doses may be used as known outputs. In an embodiment, feature vectors may be extracted from the images and used with the corresponding known outputs for the training set as inputs. A feature vector may include any suitable features (e.g., pixel intensity, etc.).

In one example, the comparable angiographic image from the second set may be identical or nearly identical to a counterpart image in the first set, other than the difference in angiographic doses. For instance, the same object may be imaged in both angiographic images, and may have substantially the same size, position and orientation in both images. The angiographic image from the first set and the counterpart image from the second set may be similar enough to provide useful training data to the deep learning neural network, e.g., data that may help train the deep learning neural network to produce a diagnostically useful angiographic image based on an angiographic image that is obtained at reduced angiographic doses and not diagnostically useful.

The training data may be acquired based on any suitable angiographic training images. For example, the training data may be obtained (1) in a laboratory setting using animals undergoing approved angiographic studies; (2) in a laboratory setting using physical models with fluid mechanically pumped into synthetic organs which are angiographically imaged; and/or (3) from full-quality human clinical angiographic data that have been computationally modified to simulate lower chemical contrast and/or x-ray doses. The data training system may use these training options alone or in any suitable combination to train the deep learning neural network.

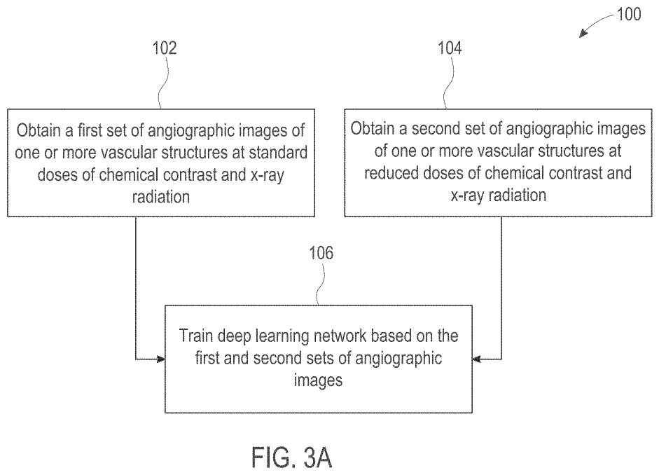

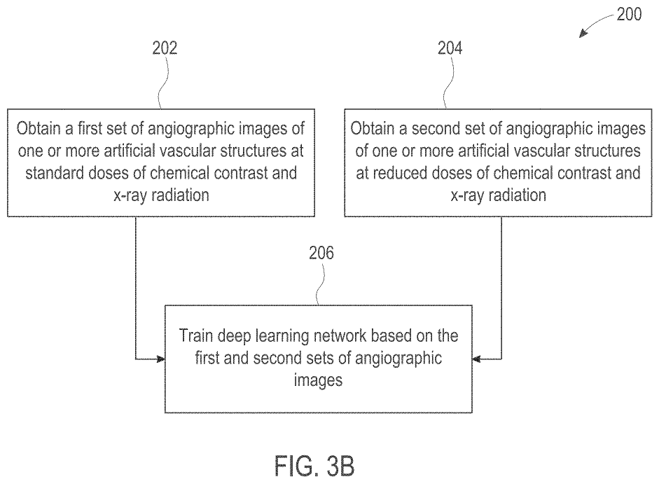

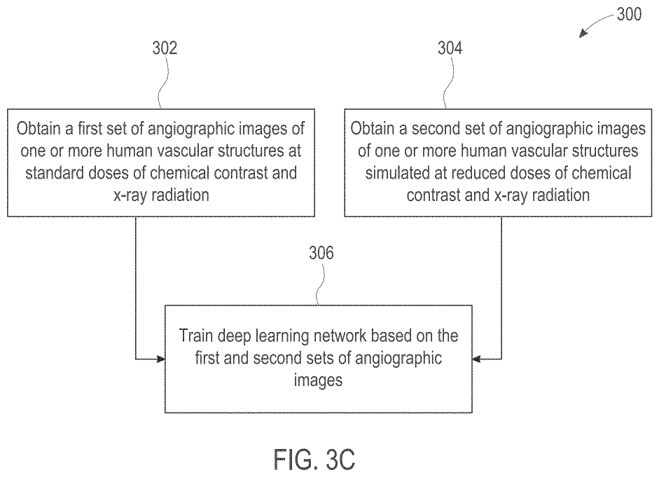

FIG. 3A illustrates a flowchart of an example method 100 for training a deep learning neural network based on vascular structures, such as non-human vascular structures (e.g., training data option (1)—training data obtained in a laboratory setting using animals undergoing approved angiographic studies). In this example, at step 102, a first set of angiographic images of one or more non-human vascular structures is obtained at standard/conventional doses of chemical contrast (e.g., for the coronary arteries, approximately 10 ml of 300 mg iodine/ml) and x-ray radiation (e.g., for an angiogram, a dose area product of approximately 400 dGyxcm²); at step 104, a second set of angiographic images of one or more non-human vascular structures is obtained at reduced doses of chemical contrast (e.g., ¼ of standard iodinated contrast doses) and/or x-ray radiation (e.g., ½ of standard radiation doses); and, at step 106, the deep learning neural network is trained based on the first and second sets.

In one example, laboratory animals may be used to obtain angiograms at conventional doses of chemical contrast and x-ray radiation in order to provide a “gold standard” reference set of angiographic images. Then, without moving the animal or the gantry position of the fluoroscopic imaging unit, angiographic studies may be obtained at lower chemical contrast and/or x-ray doses.

The angiographic images obtained based on conventional doses may be manually employed by human technicians to segment the blood vessels. These segmentations may serve as guides for the segmentations of blood vessels that are poorly seen in the stress angiograms that are similar or identical in every way except for being obtained at lower chemical contrast and x-ray doses. As used herein, the term “to segment” may refer to manually, semi-automatically, or fully-automatically identifying and representing (e.g., displaying) the vascular elements of an angiogram. A vascular “segmentation” may refer to processing of an image such that the vascular structures are represented as distinct from the noise and other structures in the imaged field of view. For example, the vascular structures in a “segmented” angiographic image may be represented as white objects against a black background. Angiographic images subject to segmentation may be referred to herein as “segmented angiographic images.” In an embodiment, segmented angiographic images obtained at conventional doses of chemical contrast and x-ray radiation may be used as training data (e.g., known outputs) with reference to the angiographic images obtained at lower doses.

FIG. 3B illustrates a flowchart of an example method 200 for training a deep learning neural network based on artificial vascular structures (e.g., training option (2)—training data obtained in a laboratory setting using physical models with fluid mechanically pumped into synthetic organs which are angiographically imaged). In this example, at step 202, a first set of angiographic images of one or more artificial vascular structures is obtained at standard doses of chemical contrast and x-ray radiation; at step 204, a second set of angiographic images of one or more artificial vascular structures is obtained at reduced doses of chemical contrast and x-ray radiation; and, at step 206, the deep learning neural network is trained based on the first and second sets.

The artificial vascular structures may be human-manufactured, solid vascular organ phantoms or organoids which include mechanical fluid pumps that simulate pulsatile arterial flow. The fluid may be pumped into the artificial vascular structure with a network of hollow tubular structures that share the size, shape, and branching pattern of the blood vessels. One manufacturer of artificial vascular structures is Heartroid, JMC Corporation, Yokohama, Japan. The artificial vascular structure may offer a suitable generator of systematic standard and low chemical contrast and x-ray dose images.

In one specific example, the organoid may be positioned in an angiographic imaging suite; fluid with chemical contrast may be injected; and fluoroscopic x-ray images may be obtained to generate a sequence of angiographic images of the chemical contrast flowing through the organoid vascular channels. Then, without changing the position of the organoid or of the imaging gantry, an interacting range of lower chemical contrast doses crossed with lower x-ray doses may be employed to obtain a large body of angiographic images at varying chemical contrast and x-ray doses. The angiographic images obtained at higher doses may serve as training data with reference to the angiographic images obtained at lower doses. Thus, the neural network may be offered the low chemical contrast and x-ray dose data for training with the vascular data obtained from the higher dose angiographic studies.

The images obtained at higher doses may be refined by human editing or by mathematical processing, e.g., using techniques discussed in A. F. Frangi, W. J. Niessen, K. L. Vincken, and M. A. Viergever, “Multiscale vessel enhancement filtering,” Lecture Notes in Computer Science (including sub-series Lecture Notes in Artificial Intelligence and Lecture Notes in Bioinformatics), vol. 1496, p. 130-137, 1998, which is hereby incorporated by reference in its entirety.

FIG. 3C illustrates a flowchart of an example method 300 for training a deep learning network based on human vascular structures (e.g., training option (3)—training data obtained by from full-quality human clinical angiographic data that have been computationally modified to simulate lower chemical contrast and x-ray doses). In this example, at step 302, a first set of angiographic images of one or more human vascular structures is obtained at standard doses of chemical contrast and x-ray radiation; at step 304, a second set of angiographic images of one or more human vascular structures is obtained which is simulated at reduced doses of chemical contrast and x-ray radiation; and, at step 306, the deep learning network is trained based on the first and second sets.

In one example, anonymized human angiographic data may be obtained at the standard chemical contrast and x-ray doses that are currently used to generate high quality angiographic images. Vascular structures may be extracted from these images by human editing and/or mathematical vessel structure filters. Then, the angiographic images may be computationally modified to simulate lower chemical contrast crossed by lower x-ray doses, e.g., based on computational methods described in M. Elhamiasl and J. Nuyts, “Low-dose x-ray ct simulation from an available higher-dose scan.” Phys Med Biol, vol. 65, no. 13, p. 135010, 2020 07 08, which is hereby incorporated by reference in its entirety.

The simulation of reduced x-ray radiation dose may be generated by adding a mixture of Poisson and Gaussian noise to the data gathered by the x-ray detector. The greater the added noise, the lower the simulated x-ray dose. To simulate lower chemical contrast, an angiographic image may be characterized by a histogram of pixel values. By treating an angiogram as normalized so that greater attenuation of x-rays by the imaged tissue is rendered as greater brightness, when a contrast bolus passes through the vasculature being imaged, the pixel histogram shifts to the right as there are more pixels with brighter values because of the contrast in the imaged blood vessels; accordingly, lower chemical contrast doses may be simulated by applying Poisson and Gaussian noise to the pixels that shift to the right in the histogram as the contrast bolus passes through. For example, a pre-contrast image of the angiographic field may be obtained using a full dosage of x-ray radiation without chemical contrast agent (e.g., before a chemical contrast agent is administered), and a post-contrast angiographic image of the same angiographic field may be obtained using full dosages of a chemical contrast agent and x-ray radiation (e.g., after a chemical contrast agent is administered). For corresponding pixels in the two images, a difference in brightness or intensity between the two corresponding pixels in the images may be determined (e.g., by subtracting their respective pixel values). The brightness difference between the two corresponding pixels may be stored (e.g., as a floating point number between 0 and 1) in a first matrix, and a second matrix of random numbers (e.g., floating point numbers between 0 and 1 randomly generated using a Poisson or Gaussian distribution) may be generated which corresponds in size to the first matrix. For each non-zero cell in the first matrix, the random number in the corresponding cell of the second matrix may be subtracted from the non-zero cell. The results may be stored in a third matrix which may then be added to the pixel values in the pre-contrast image to obtain a simulated reduced-dosage angiographic image.

To avoid registration error due to movement of a vessel in the angiographic field of view before and after administration of the chemical contrast agent, pixels representing the vessel may be identified, and a greater quantity of random noise may added to these pixels to simulate a low dose of chemical contrast agent. In particular, the noise may have a negative mean to simulate a lowering of signal from lower contrast, and a high standard deviation to simulate lowering of signal noise. Using DICOM pixel values of 0-32,000 as an example (instead of floating point numbers between 0 and 1), suppose that the mean pixel value in an image before the chemical contrast agent arrives is a first number (e.g., 50±10). After the chemical contrast agent arrives, some pixels turn brighter because they represent vessels containing the chemical contrast agent. Thus, the mean pixel value increases overall (e.g., to 60±15). In such a case, pixel values greater than the pre-contrast mean value plus two standard deviations (e.g., 50+20) may be assumed to be vessel pixels (i.e., pixels that represent vessels). Thus, a greater quantity of random noise may be added specifically to the vessel pixels to simulate lower contrast dose.

Any suitable data preparation operations may be applied for simulated dose reduction computational transforms. In one example, one or more data augmentation operations may be performed to reduce over-fitting in the deep learning neural network. Examples of such data augmentation operations may include arbitrary translations and rotations of the training data. In one example, the simulated dose reduction transforms may be incorporated into a data augmentation step.

The simulated lower dose images, which preferably have worse signal-to-noise ratios than the unmodified images, may be provided to the neural network. The neural network may, in turn, use the vascular structures obtained from the images taken with standard chemical contrast and x-ray doses as reference vascular data (i.e., a known output) that the network is trained to detect and produce. Thus, the simulated lower dose data may be used as an input to train a deep neural network to produce angiographic images comparable to those obtained from the full chemical contrast and x-ray dose data. In an embodiment, the reference vascular data may include segmented angiographic images obtained at full dosages of chemical contrast and x-ray radiation to train the deep neural network to produce high-quality segmented angiographic images from corresponding non-segmented angiographic images obtained or simulated to have been obtained at reduced dosages of chemical contrast and/or x-ray radiation.

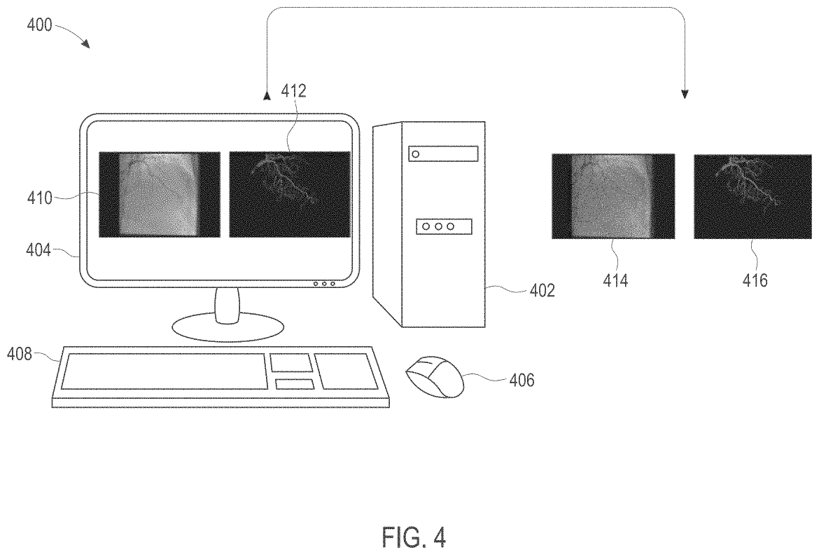

FIG. 4 illustrates an example system 400 configured to generate images of vascular structures simulated at lower doses of chemical contrast and x-ray radiation, according to an example embodiment. The example system 400 includes a computer 402, a display 404 connected to the computer, a pointing device 406 such as a mouse or trackpad connected to the computer, and a keyboard 408 connected to the computer. Computer 402 may include a processor in communication with memory and/or other non-transitory data storage devices containing instructions executable by the processor to segment angiographic images. The computer 402 may also store instructions executable by the processor to operate a neural network in training and/or deployment modes. Computer 402 may also have a communications interface configured to send and receive angiographic data over a communications network such as a local area network or a wide area network or the like. The system 400 may be leveraged for multi-frame deep learning. In one example, a human coronary artery angiogram is obtained. In this example, angiography of the human heart is employed; however, these techniques may apply to angiography of other organs or animals. In the example of FIG. 4, the neural network may be operating in a training mode.

Angiographic images stored on the computer 402 may be displayed on the display 404 of the computer system 400. An example coronary angiogram image 410 is shown being displayed on the display 404. Computer system 400 may obtain image 410 in any conventional manner. For example, the image 410 may be obtained directly from an angiographic imaging system, such as the system shown in FIGS. 1A and 1B, over a hardwired connection, a wireless connection, or a communications network. In another example, computer system 400 may obtain image 410 from a remote source via a local area network, a wide area network, or some other type of communications network. In yet another example, computer system 400 may upload the image from a portable data storage device such as a USB thumbdrive, a DVD, or the like. In one example, a human analyst examines the example image 410 and interacts with the system by employing a graphical user interface device such as a mouse 406 to select (i.e., paint) the pixels that represent blood vessels in a segmented coronary angiogram image 412. In certain examples, segmenting may be performed purely by a human analyst by painting over an angiographic image. In other examples, a mathematical or deep learning segmentation algorithm may make an initial guess at the segmentation. In still other examples, the mathematical or deep learning segmentation algorithm may perform the painting autonomously.

FIG. 4 further illustrates simulated dose reduced coronary angiogram image 414, which has a simulated reduction of x-ray dose compared to the angiographic image 410. The angiographic image 410 may be used to generate a simulated dose reduced coronary angiogram image 414 using any suitable techniques for simulating reduced x-ray radiation doses, as discussed above. For example, computer system 400 may be used to generate the simulated dose reduced coronary angiogram image. Once generated, simulated dose reduced coronary angiogram image 414 may be employed for training of the deep learning neural network by reference to the segmentation of the same angiographic image obtained at full x-ray dose at conserved segmented coronary angiogram image 416 (which may be the same image as segmented image 412).

In FIG. 4, the angiographic image 410 and the segmented image 412 are shown being displayed side by side, but in other examples the painting may take place on the same image on the display 404. Thus, the painting may be overlaid on the angiographic image 410 to generate the segmented image 412.

In training mode, the neural network may obtain, as training data, a plurality of temporally consecutive or contiguous images (e.g., five images) obtained at standard doses and a plurality of corresponding images obtained at (or simulated to have been obtained at) low doses. The images may be selected from a Digital Imaging and Communications in Medicine (DICOM) file including, e.g., approximately eighty total images. Each image may have a size of 512×512 pixels, and each pixel may be represented in DICOM format as an integer (e.g., between approximately 0 and approximately 16,000).

In an embodiment, one or more of the temporally consecutive or contiguous images (e.g., five images) obtained at standard doses may be used as a known output for the neural network, and one or more of the plurality of corresponding images obtained at (or simulated to have been obtained at) low doses may be used to encode the input layer of the neural network. Based on the training data, the neural network may form connections between individual neurons, each connection having one or more associated weights (e.g., floating point numbers) to produce the known output from the input. In an embodiment, if the known output comprises a segmented angiographic image, the neural network may be trained to assign, to each pixel in the output image, a floating point number between 0 and 1 representing the probability of the pixel being part of a blood vessel. In another embodiment, the neural network may assign, to each pixel, a plurality of floating point numbers between 0 and 1, inclusive, wherein each floating point number represents a probability of the pixel being part of a certain feature (e.g., a blood vessel, a catheter, and/or a branch point). For example, three floating point numbers between 0 and 1 may be assigned, wherein one floating point number may represent the probability of the pixel being part of a blood vessel; another floating point number may represent the probability of the pixel being part of a catheter; and another floating point number may represent the probability of the pixel being part of a branch point (e.g., where one blood vessel splits into two or more blood vessels).

A checkpoint file that stores weights for the neural network may be used in a deployment/prediction mode. For example, training with images obtained at standard angiographic doses may result in a first checkpoint file with one set of weights, and training with images obtained at (or simulated to have been obtained at) lower angiographic doses may result in a second checkpoint file with a different set of weights. The first checkpoint file may be applied to an input of one or more images obtained at standard angiographic doses to obtain one or more segmented images, and the second checkpoint file may be applied to an input of one or more images obtained at (or simulated to have been obtained at) lower angiographic doses to obtain one or more segmented images. The second checkpoint file may be applied in a clinical setting where lower angiographic doses are administered to spare the patient adverse side effects of higher angiographic doses while conserving angiographic image quality.

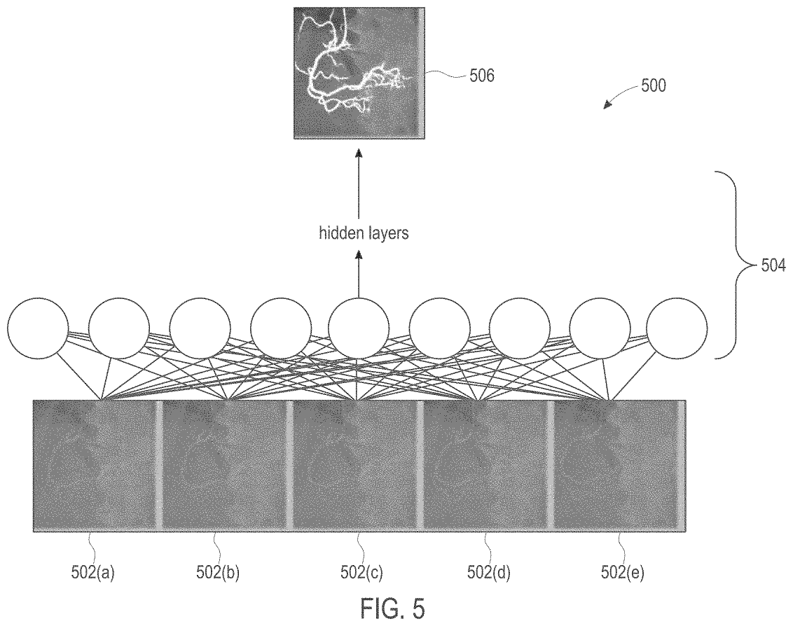

FIG. 5 illustrates an example method 500 for segmenting vasculature objects in a single image from an angiogram acquired at low chemical contrast or x-ray doses. In this example, a convolutional network draws information from several angiographic images to perform the segmentation. In the example of FIG. 5, the neural network may be operating in a deployment mode.

Analyzing (e.g., performing calculations on) an entire sequence of angiographic images may exceed practical computer memory and computational speed limits; thus, as described in connection with FIG. 5, the techniques described herein may enable generation of a high-quality image based on a sub-sequence of images within the relevant computer memory and computational speed constraints. As used herein, the term “sequence” may indicate an entire set of angiographic images, e.g., images that are fluoroscopically acquired across the travel of the injected contrast bolus. The term “sub-sequence” may indicate a subset of the sequence of images that are provided to a deep learning neural network system to estimate the vascular structure. Angiographic images in a “sub-sequence” are preferably temporally contiguous or consecutive, but may be separated by intervening images.

FIG. 5 demonstrates the implementation of the vessel segmentation of a single angiographic image from a sub-sequence of angiographic images that are noisy because they were acquired with low chemical contrast or low x-ray radiation doses. In this example, the angiographic image that is segmented is drawn from a sub-sequence of five temporally adjacent angiographic images 502(a)-(e), which are drawn from physically reduced chemical contrast and x-ray radiation dose angiograms. In this example, the middle (third) image 502(c) in the sub-sequence of five angiographic images is the target image (image of attention). The target image may be the image for which the neural network is to generate a higher-quality version.

The sub-sequence of the five angiographic images 502(a)-(e) may be combined and supplied to a convolutional neural network 504 as a single input. For example, if each image comprises a 512×512 vector of pixel values, the five angiographic images may be inputted or encoded to the neural network as a 5×512×512 vector of pixel values, and the neural network may be trained to produce a single high-quality and/or segmented angiographic image corresponding to one of the five angiographic images (e.g., the middle image 502(c)).

The convolutional neural network 504 may have been previously trained with simulated low-dose, noisy images against vessel segmentations extracted from the standard-dose, non-noisy images (e.g., as discussed above in relation to FIG. 4). For example, to train the neural network to produce a single high-quality and/or segmented angiographic image from five lower-quality temporally contiguous images, a sub-sequence of lower-quality images containing a target image may be obtained. The target image is preferably located in a middle of the sub-sequence so that there are one or more images before the target image and one or more images after the target image. In this way, the neural network may be trained to use spatiotemporal information in providing an output. In the sub-sequence, the number of images before the target image may differ from the number of images after the target image. However, in a preferred embodiment, the target image is centrally located in the sub-sequence so that there are an equal number of images on each side of the target image. For example, in the case of five temporally contiguous images, the target image is preferably located between two temporally contiguous images on each side of the target image. In an embodiment, the neural network may be trained with a plurality of sub-sequences. For example, if there are 10 images from a first angiogram and 12 images from a second angiogram available, a training set may be assembled with up to fourteen sets of unique sub-sequences in which each subsequence consists of five temporally contiguous images. That is, six unique sub-sequences of five temporally contiguous images can be assembled from the first angiogram (because the third through eighth images are each located in the middle of five temporally contiguous images), and eight sub-sequences of five temporally contiguous images can be assembled from the second angiogram (because the third through tenth images are each located in the middle of five temporally contiguous images). The first, second, penultimate, and last images in each series are not targets in this example because they are not located in the middle of five temporally contiguous images. While it has been found that using sub-sequences of five temporally contiguous images is advantageous in that it allows for a high-quality and/or segmented image to be produced with less processing and relatively few images at ends of the sequence not themselves being targets, present techniques may be adapted to use sub-sequences containing fewer than five temporally contiguous images or more than five temporally contiguous images. In general, increasing the number of images in a sub-sequence will tend to increase the resulting signal-to-noise ratio the images produced by the neural network. At the same time, increasing the number of images in a sub-sequence increases the number of images dropped-off at the ends.

While it is advantageous to use an odd number of temporally contiguous images as a sub-sequence for training a neural network to produce a high-quality and/or segmented image corresponding to a target image at the center of the subsequence (e.g., because it is based on an equal amount of spatiotemporal information before and after the target image), the present techniques may be adapted to produce a high-quality and/or segmented image corresponding to a target image that is not at the center of the sub-sequence (e.g., the first, second, fourth, or fifth image of a five-image sub-sequence).

The present techniques may also be adapted to use sub-sequences consisting of an even number of temporally contiguous images. An advantage to using sub-sequences consisting of an even number of temporally contiguous images is that it may permit greater flexibility in the location of the target image and the size of the sub-sequence (e.g., allowing the first or last image in a sequence of images to be targeted using only a sub-sequence of only two temporally contiguous images). It will also be appreciated that the present techniques may be adapted to use a combination of odd and even-numbered sub-sequences to produce high-quality and/or segmented images corresponding to corresponding targets located anywhere in the sequence. In another embodiment, a plurality of neural networks may be deployed in which each neural network is trained with a different number of input images, ranging from as few as one image up to as many images as processing resources allow. The use of a plurality of neural networks in this manner allows high-quality and/or segmented images to be produced from every image in an angiogram.

In an embodiment, images in a training set may be subjected to a data augmentation step in which, e.g., images are translated and/or rotated prior to being inputted to train the neural network to generalize better. It will be appreciated that a simulation of reduced dosages of chemical contrast agent and/or x-ray radiation may be incorporated as part of the data augmentation step.

Because it is operating on the sub-sequence of five noisy angiographic images 502(a)-(e), the trained convolutional neural network 504 may estimate a single segmented image 506 that has a greater signal-to-noise ratio than if the trained convolutional neural network were operating on only a single noisy angiographic image. The segmented image 506 may represent the deep learning neural network estimation of the vascular structure of the middle (third) image in the sub-sequence of five noisy angiographic images 5(a)-(e).

More specifically, the neural network 504 may load the floating point numbers from the checkpoint file generated during the training mode, and, based on the floating point numbers, output the segmented image 506 from the five noisy angiographic images 5(a)-(e). The neural network may generate, as output, one or more of three images: one image based on the probability of pixels being part of a blood vessel; another image based on the probability of the pixels being part of a catheter; and another image based on the probability of the pixel being part of a branch point. These three images may be stacked/overlaid to generate the segmented image 506 showing blood vessels, a catheter, and/or branch points in high-quality, e.g., as if the target image was obtained at standard angiographic doses.

In one example, the convolutional neural network 504 used for angiographic segmentation may be based on a U-Net architecture, e.g., as described in O. Ronneberger, P. Fischer, and T. Brox, “U-net: Convolutional networks for biomedical image segmentation,” in Medical Image Computing and Computer-Assisted Intervention—MICCAI 2015, N. Navab, J. Hornegger, W. M. Wells, and A. F. Frangi, Eds. Cham: Springer International Publishing, 2015, pp. 234-241, which is hereby incorporated by reference in its entirety.

In one specific example, source code for the deep learning neural network may be written in Python language and generated as a U-net structure in the Pytorch machine learning software library (https://pytorch.org, which is hereby incorporated by reference in its entirety). The U-net structure may be three-dimensional in the sense that each image has two spatial dimensions, and the vasculature within the images may be estimated from both the image and temporally nearby angiographic images. In one specific example, the Pytorch library for neural network machine learning may include a Python base class named nn.Module, as documented at https://pytorch.org/docs/stable/generated/torch.nn.Module.html, which is hereby incorporated by reference in its entirety. All neural network modules may comprise subclass nn.Module. A python class named UNet may inherit from nn.Module. The Python class Unet may include the convolutional neural net structure with spatiotemporal properties. It will be appreciated that the techniques described herein may be implemented using any suitable machine learning mechanism.

It will be appreciated that any suitable programming languages, libraries, toolsets, and/or other mechanisms may be used for developing a deep learning neural network in accordance with example provided herein. For example, the U-net structure may be extended to a full three-dimensional structure that simultaneously estimates the vascular structures in the plurality of the temporally adjacent angiographic images.

The convolutional neural network 504 may have the structure of an encoder-decoder. The convolutional neural network 504 may also include jump connections between layers of the same size on the encoder and the decoder. These jump connections may enable the output of segmented image 506 with a similar degree of granularity as the angiogram sub-sequence inputs (e.g., the sub-sequence of the five angiographic images 502(a)-(e)). The loss function used to train this architecture may be a linear combination of a classification loss-function, cross-entropy, and a dice loss function for crisp boundary detection.

To prevent the movement of blood vessels between angiographic image frames from interfering with the angiographic data, the structure of convolutional neural network 504 may have a high spatiotemporal convolutional density. That is, an organ that experiences larger motion, such as the heart, may be imaged at 15 Hz, with a neighborhood of five images being used to determine the vasculature present in the middle image; an organ with less motion, such as the brain, may be imaged at, e.g., 6 Hz, with a neighborhood of five images being used to determine the vasculature present in the middle image. Thus, the convolutional neural network 504 may account for both lesser motion (such as motion in the brain, where motion is limited by the surrounding rigid container of the cranial bone) as well as greater motion (such as motion in the heart, which is a muscular organ that is continually beating to pump blood into an arterial system).

In a deep learning network training mode (in contrast to the deployment mode illustrated in FIG. 5), the roles of the data sources (inputs) and products (outputs) may be altered. For example, the image 506 may represent a ground truth representation of the vasculature structure as would be obtained from full dose chemical contrast and x-ray radiation dose angiography, and the one or more images of the sub-sequence 502(a)-(e) may be obtained from empirically reduced dose studies from animal or physical organoid model angiography, or computationally simulated reduced images drawn from the ground truth full dose image 506. In training mode, training may occur based on both the one or more images of the sub-sequence 502(a)-(e) and the segmented image 506.

While FIG. 5 illustrates the sub-sequence 502(a)-(e) as having five angiographic images, it will be appreciated that the quantity of five is simply an example. In some circumstances, fewer or greater than five images may be employed, even within the same angiographic study. For example, more than five angiographic images may be used to estimate the segmentation of the middle image of an angiographic sequence containing dozens of individual images. This may increase the signal-to-noise ratio in the angiographic image of interest compared to using only five angiographic images (but at the cost of increased computational resources), thereby allowing for further reduction in chemical contrast and/or x-ray radiation doses.

In such circumstances, the vessel segmentation of angiographic images toward the beginning or the end of the sequence may be estimated based on fewer surrounding images. For example, the sixth image may be segmented based on its position in the middle of five images (e.g., numbers 4, 5, 6, 7, and 8), whereas the fifth image cannot be in the middle of a sub-sequence of five images, but could be treated as being in the middle of a sub-sequence of three images (e.g., numbers 4, 5, and 6).

In addition, there may be circumstances where the angiographic image of attention (e.g., the target image) is not in the middle of the sub-sequence, but is instead in another position (e.g., near the beginning or the end of the sub-sequence). For example, during angiographic catheter positioning maneuvers, the angiography physician may choose to perform intermittent real-time angiographic contrast study injections while acquiring fluoroscopic images. The angiographic image of interest cannot be a middle image in the context of real-time angiographic imaging because the future angiographic images have not yet been acquired. Instead, a deep learning network may perform the signal to noise enhancement based on the target image and a temporally preceding plurality of images.

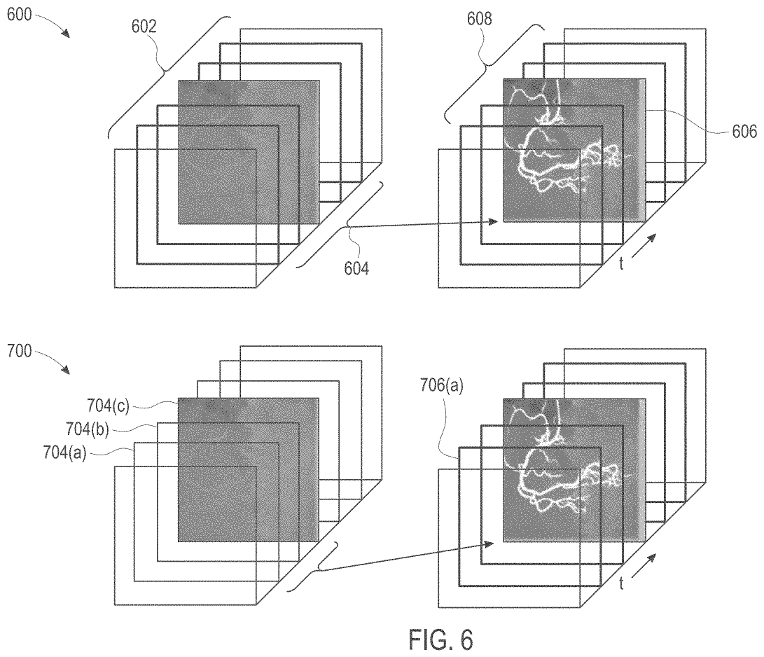

FIG. 6 depicts the sub-setting of a sequence of angiographic images into a plurality of overlapping sub-sequences where each estimates the vascular structure for one angiographic image. More specifically, FIG. 6 illustrates a first example method for estimating a higher-quality image in which a target image is in the middle of a sub-sequence, and a second example method for estimating a higher-quality image in which a target image that is not in the middle of a sub-sequence. The first method may apply to offline fluoroscopic angiography, and the second method may apply to real-time fluoroscopic angiography.

The first method 600 is illustrated in the top panel of FIG. 6. As shown, a sequence of angiographic images 602, of total length n, is produced. Within the sequence of angiographic images 602, there is a sub-sequence of five angiographic images 604 of length five where the image of attention for segmentation by the deep learning network is in the middle. The neural network may produce a segmented image 606 that corresponds to the middle (third) angiographic image in the sub-sequence. This process may be incremented by one image for the sequence of angiographic images 602 until every image but the first two and the last two have been the image of attention for deep learning segmentation. This may produce a sequence of segmented images 608.

The second method 700 is illustrated in the bottom panel of FIG. 6. Here, the angiographic image of attention may be the first (most recent) angiographic image 704(a) of a sub-sequence of angiographic images 704(a)-(c). The neural network may apply a deep learning calculation to improve the signal-to-noise ratio of the target image 704(a) to produce a high-quality segmented version 706(a) of the target image, and thereby to improve the tolerance of the image quality to reductions in chemical contrast dose and x-ray radiation dose. For example, the neural network may use the target image and one or more subsequent, temporally contiguous images as the inputs for the deep learning network calculation.

The present invention may include a method, system, device, and/or a computer program product at any possible technical detail level of integration. The computer program product may include a computer readable storage medium (or media) having computer readable program instructions thereon for causing a processor to carry out aspects of the present invention.

Computer readable program instructions described herein can be downloaded to respective computing/processing devices from a computer readable storage medium or to an external computer or external storage device via a network, for example, the Internet, a local area network, a wide area network and/or a wireless network. The network may comprise conductive transmission cables, optical transmission fibers, wireless transmission, routers, firewalls, switches, gateway computers and/or edge servers. A network adapter card or network interface in each computing/processing device may receive computer readable program instructions from the network and forward the computer readable program instructions for storage in a computer readable storage medium within the respective computing/processing device.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a non-transitory computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The above description is for the purpose of teaching the person of ordinary skill in the art how to practice the subject of the present application, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

What is claimed is:

1. A method comprising:
providing, as an input to a machine learning model, via a processor, a target angiographic image obtained using a first dose of a chemical contrast agent and/or x-ray radiation, the machine learning model having been trained using (a) a second angiographic image obtained using a second dose of a chemical contrast agent and/or x-ray radiation as an input, and (b) a third angiographic image obtained using a third dose of a chemical contrast agent and/or x-ray radiation as a known output, wherein the third dose is greater than the first and second doses; and
obtaining, from the machine learning model, via the processor, an output comprising an angiographic image that is a processed version of the target angiographic image.

2. The method of claim 1, wherein the target image is one of a plurality of angiographic images in a first sub-sequence of angiographic images obtained using the first dose of a chemical contrast agent and/or x-ray radiation and provided to the machine learning model as an input, wherein the second angiographic image is one of a plurality of angiographic images in a second sub-sequence of angiographic images obtained using the second dose of a chemical contrast agent and/or x-ray radiation and used to train the machine-learning model.

3. The method of claim 2, wherein the first sub-sequence of angiographic images is provided to the machine learning model as a single vector.

4. The method of claim 2, wherein the first and second sub-sequences of angiographic images each consist of an odd number of angiographic images.

5. The method of claim 4, wherein the target image is in a middle of the first sub-sequence of angiographic images.

6. The method of claim 2, wherein the first subsequence of angiographic images consists of a same number of angiographic images as the second sub-sequence of angiographic images.

7. The method of claim 2, wherein the first and second sub-sequences each consist of five angiographic images.

8. The method of claim 2, wherein the first sub-sequence of angiographic images is one of a plurality of input sub-sequences, wherein each of the plurality of input sub-sequences was extracted from a different part of a sequence of angiographic images obtained using the first dose of a chemical contrast agent and/or x-ray radiation, and further comprising providing, via the at least one processor, the plurality of input sub-sequences to the machine learning model as inputs and obtaining, via the at least one processor, from the machine learning model, outputs comprising angiographic images that are processed versions of respective target angiographic images in each of the plurality of input sub-sequences.

9. The method of claim 2, wherein the second sub-sequence of angiographic images is one of a plurality of sub-sequences used to train the machine-learning model, and wherein each of the plurality of sub-sequences was extracted from a different part of a sequence of angiographic images obtained using the second dose of a chemical contrast agent and/or x-ray radiation.

10. The method of claim 2, wherein each of the angiographic images in the first sub-sequence of angiographic images is temporally contiguous with at least one other angiographic image in the first sub-sequence, and wherein each of the angiographic images in the second sub-sequence of angiographic images is temporally contiguous with at least one other angiographic image in the second sub-sequence.

11. The method of claim 1, wherein the first and second doses are the same.

12. The method of claim 1, wherein the first and second doses are less than a dose required to obtain diagnostically useful images from an x-ray imaging device, and wherein the third dose is no less than the dose required to obtain diagnostically useful images from the x-ray imaging device.

13. The method of claim 2, wherein the angiographic images in the second sub-sequence are angiographic images of non-human vascular structures.

14. The method of claim 2, wherein the angiographic images in the second sub-sequence are angiographic images of artificial vascular structures.

15. The method of claim 2, wherein the second dose of chemical contrast agent and/or x-ray radiation is greater than the first dose of chemical contrast agent and/or x-ray radiation, and wherein the angiographic images in the second sub-sequence are angiographic images that have been modified to simulate having been obtained using less than the second dose of chemical contrast agent and/or x-ray radiation.

16. The method of claim 1, wherein the second dose of chemical contrast agent and/or x-ray radiation is greater than the first dose of chemical contrast agent and/or x-ray radiation, and wherein the second angiographic image has been modified to simulate having been obtained using less than the second dose of chemical contrast agent and/or x-ray radiation.

17. The method of claim 16, wherein the second angiographic image is modified by adding randomly generated noise to at least some pixels of the second angiographic image.

18. The method of claim 17, wherein the randomly generated noise is added only to pixels of the second angiographic image that are determined to correspond to vessels.

19. The method of claim 1, wherein the third angiographic image is a segmented angiographic image, and wherein the output obtained from the machine learning model is an angiographic image that is a segmented version of the first angiographic image.

20. The method of claim 1, further comprising displaying, via the processor, the processed version of the first angiographic image on a display.

21. A system comprising:
one or more memory devices; and
at least one processor coupled to the one or more memory devices, the at least one processor configured to:
provide, as an input to a machine learning model, a target angiographic image obtained using a first dose of a chemical contrast agent and/or x-ray radiation, the machine learning model having been trained using (a) a second angiographic image obtained using a second dose of a chemical contrast agent and/or x-ray radiation as an input, and (b) a third angiographic image obtained using a third dose of a chemical contrast agent and/or x-ray radiation as a known output, wherein the third dose is greater than the first dose; and
obtain, from the machine learning model, an output comprising an angiographic image that is a processed version of the target angiographic image.

22. The system of claim 21, wherein the target image is one of a plurality of angiographic images in a first sub-sequence of angiographic images obtained using the first dose of a chemical contrast agent and/or x-ray radiation, wherein the second angiographic image is one of a plurality of angiographic images in a second sub-sequence of angiographic images obtained using the second dose of a chemical contrast agent and/or x-ray radiation and used to train the machine-learning model, and wherein the at least one processor is configured to provide the plurality of angiographic images in the first sub-sequence to the machine learning model as the input.

23. The system of claim 22, wherein the target image is in a middle of the first sub-sequence of angiographic images.

24. The system of claim 22, wherein the first sub-sequence of angiographic images is one of a plurality of input sub-sequences of angiographic images provided to the machine-learning model, and wherein each of the plurality of input sub-sequences was extracted from a different part of a sequence of angiographic images obtained using the first dose of a chemical contrast agent and/or x-ray radiation, and wherein the processor is configured to provide the plurality of input sub-sequences to the machine learning model as inputs to obtain, from the machine learning model, outputs comprising angiographic images that are processed versions of respective target angiographic images in each of the input sub-sequences.

25. The system of claim 22, wherein the first sub-sequence of angiographic images consists of a same number of angiographic images as the second sub-sequence of angiographic images.

26. The system of claim 21, wherein the third dose of chemical contrast agent and/or x-ray radiation is greater than the second dose of chemical contrast agent and/or x-ray radiation.

27. The system of claim 21, wherein the second dose of chemical contrast agent and/or x-ray radiation is greater than the first dose of chemical contrast agent and/or x-ray radiation, and wherein the second angiographic image has been modified to simulate having been obtained using less than the second dose of chemical contrast agent and/or x-ray radiation.

28. The system of claim 21, wherein the first and second doses are less than a dose required to obtain diagnostically useful images from an x-ray imaging device, and wherein the third dose is no less than the dose required to obtain diagnostically useful images from the x-ray imaging device.

29. The system of claim 21, wherein the third angiographic image is a segmented angiographic image, and wherein the output obtained from the machine learning model is an angiographic image that is a segmented version of the first angiographic image.

30. The system of claim 21, wherein the system further comprises a display device, and wherein the processor is configured to display the processed version of the first angiographic image on the display device.

31. A computer program product comprising one or more non-transitory computer readable media having instructions stored thereon, the instructions executable by at least one processor to cause the at least one processor to:
provide, as an input to a machine learning model, a target angiographic image obtained using a first dose of a chemical contrast agent and/or x-ray radiation, the machine learning model having been trained using (a) a second angiographic image obtained using a second dose of a chemical contrast agent and/or x-ray radiation as an input, and (b) a third angiographic image obtained using a third dose of a chemical contrast agent and/or x-ray radiation as a known output, wherein the third dose is greater than the first dose; and
obtain, from the machine learning model, an output comprising an angiographic image that is a processed version of the target angiographic image.

32. The computer program product of claim 31, wherein the target image is one of a plurality of angiographic images in a first sub-sequence of angiographic images obtained using the first dose of a chemical contrast agent and/or x-ray radiation, wherein the second angiographic image is one of a plurality of angiographic images in a second sub-sequence of angiographic images obtained using the second dose of a chemical contrast agent and/or x-ray radiation and used to train the machine-learning model, and wherein the instructions are executable by the at least one processor to cause the at least one processor to provide the plurality of angiographic images in the first sub-sequence to the machine learning model as the input.

33. The computer program product of claim 32, wherein the target image is in a middle of the first sub-sequence of angiographic images.

34. The computer program product of claim 32, wherein the first sub-sequence of angiographic images is one of a plurality of input sub-sequences of angiographic images provided to the machine-learning model, and wherein each of the plurality of input sub-sequences was extracted from a different part of a sequence of angiographic images obtained using the first dose of a chemical contrast agent and/or x-ray radiation, and wherein the instructions are executable by the at least one processor to cause the at least one processor to provide the plurality of input sub-sequences to the machine learning model as inputs to obtain, from the machine learning model, outputs comprising angiographic images that are processed versions of respective target angiographic images in each of the input sub-sequences.

35. The computer program product of claim 32, wherein the first sub-sequence of angiographic images consists of a same number of angiographic images as the second sub-sequence of angiographic images.

36. The computer program product of claim 31, wherein the third dose of chemical contrast agent and/or x-ray radiation is greater than the second dose of chemical contrast agent and/or x-ray radiation.

37. The computer program product of claim 31, wherein the second dose of chemical contrast agent and/or x-ray radiation is greater than the first dose of chemical contrast agent and/or x-ray radiation, and wherein the second angiographic image has been modified to simulate having been obtained using less than the second dose of chemical contrast agent and/or x-ray radiation.

38. The computer program product of claim 31, wherein the first and second doses are less than a dose required to obtain diagnostically useful images from an x-ray imaging device, and wherein the third dose is no less than the dose required to obtain diagnostically useful images from the x-ray imaging device.

39. The computer program product of claim 31, wherein the third angiographic image is a segmented angiographic image, and wherein the output obtained from the machine learning model is an angiographic image that is a segmented version of the first angiographic image.

40. The computer program product of claim 31, wherein the instructions are executable by the at least one processor to cause the at least one processor to display the processed version of the first angiographic image on a display device.
